(19) United States
(12) Patent Application Publication
(10) Pub. No.: US 2017/0173196 A1
(43) Pub. Date: Jun. 22, 2017

(54) COMPOSITIONS COMPRISING AN ESTER AND/OR AN ACID

(71) Applicant: The Procter & Gamble Company, Cincinnati, OH (US)

(72) Inventors: Alan Edward SHERRY, Newport, KY (US); Molly Pelon BOHLEN, Montgomery, OH (US); Samuel Kimani NJOROGE, Cincinnati, OH (US); LaShanda Marie GLENN, Maineville, OH (US); Janice Lynn DHONAU, Maineville, OH (US); Christopher Todd MORRISSEY, Mason, OH (US); Jamesina Anne FITZGERALD, Trenton, OH (US); Phillip Kyle VINSON, Fairfield, OH (US); Melinda Phyllis STEFFEY, Liberty Township, OH (US); Rafael ORTIZ, Milford, OH (US); Benjamin Patrick HALE, West Chester, OH (US)

(21) Appl. No.: 15/372,433

(22) Filed: Dec. 8, 2016

Related U.S. Application Data
(60) Provisional application No. 62/271,062, filed on Dec. 22, 2015.

(51) Int. Cl. A61L 2/18; A01N 25/22; A01N 37/02; A01N 25/30; A61L 2/22; A01N 59/00

(52) U.S. Cl. CPC A61L 2/186; A61L 2/22; A01N 59/00; A01N 37/02; A01N 25/30; A01N 25/22; A61L 2202/15

(57) ABSTRACT

The present disclosure relates to compositions comprising hydrogen peroxide in combination with C₆₋₁₀ fatty acid, C₆₋₁₀ fatty acid ester derivatives, or mixtures thereof, as well as products incorporating the compositions, and methods of using the compositions and products.

### FIELD OF THE INVENTION

The present disclosure relates to compositions comprising hydrogen peroxide in combination with C₆₋₁₀ fatty acid, C₆₋₁₀ fatty acid ester derivatives, or mixtures thereof, as well as products incorporating the compositions, and methods of using the compositions and products.

### BACKGROUND OF THE INVENTION

The past decade has ushered an era of unprecedented advances in aqueous antimicrobial chemistry. Much effort has been directed at supplementing the antimicrobial activity of compositions comprising a combination of surfactant, organic acid, and either a North American registered active or a notified active recognized by the European Biocidal Products Regulation. At the same time, increased environmental awareness has created new demand for compositions based on renewable actives. The net result has been a proliferation of commercially available antimicrobial products based on organic acids, essential oils, silver compounds, and hydrogen peroxide that promise increasing cidal speed and broad spectrum activity. Given the limited number of government-accepted antimicrobial actives, advances have required the identification of safe, non-active raw materials, which, when combined with known antimicrobials, result in potentiation of antimicrobial activity.

The desire for an ever faster mode of action and broad spectrum activity continues to drive the need for hydrogen peroxide-based compositions that exhibit improved biocidal activity. An enhanced peroxycarboxylic acid composition for use at reduced concentrations and temperatures comprising: at least one C₁-C₂₂ carboxylic acid; at least one C₁-C₂₂ percarboxylic acid; hydrogen peroxide; a surfactant or a sulfonated carboxylic acid and a corresponding sulfonated percarboxylic acid; and an alcohol, where the concentration of the peroxycarboxylic acids is less than about 2000 ppm and is effective against Bacillus species at temperatures at or below about 50° is known. A germicide composition that contains (a) an active oxygen-generating compound, (b) a fatty acid ester-based surfactant and water, and the pH at 20° C. is 2-9, is known. A disinfectant composition including a peroxide, a peracid, an anionic surfactant, a nonionic polymer, and one or both of a linear fatty alcohol, and an alkyl pyrrolidone is also known.

A method of reducing a microbial population in an aqueous stream used for transporting or processing food product, the method comprising: adding to the aqueous stream a medium chain peroxycarboxylic acid composition; and reducing the microbial population in the aqueous stream; the added medium chain peroxycarboxylic acid composition comprising: about 0.5 to about 5 wt-% of peroxyoctanoic acid; about 1 to about 10 wt-% octanoic acid; about 5 to about 97 wt-% water; about 1 to about 20 wt-% of a anionic surfactant; about 6 to about 10 wt-% of H₂O₂; about 25 to about 30 wt-% inorganic acid; and about 1 to about 5 wt-% sequestrant is known. A sanitizing rinse additive composition comprising: a C₁-C₂₂ peroxycarboxylic acid; a C₁-C₂₂ carboxylic acid; hydrogen peroxide; and a nonionic defoaming and wetting surfactant(s); where the composition is a low odor concentrate having less than about 2 wt-% peroxyacetic and peracid acid is also known.

Known compositions are often effective at very low pHs, and cidal performance is often very pH dependent; it becomes increasingly difficult to maintain cidal effectiveness at pHs greater than about 2 (especially for concentrates intended to be diluted prior to use). However, low-pH compositions may be less effective for cleaning. Also, compositions based on peracetic acid (also known as peroxyacetic acid) chemistry may have an undesirable, pungent odor. Therefore, there remains a need to deliver improved antimicrobial performance and improved cleaning, especially for concentrates intended to be diluted prior to use. For ready-to-use sprays and wipe products, enhanced cidal activity versus a broad spectrum of microorganisms remains a need. There is also increasing demand for products having an increased content of components derived from renewable sources, as well as for food-safe products. It has now been found that aqueous compositions comprising one or more surfactant(s), where the surfactant comprises from about 6 to about 12 carbon atoms, an acidifying agent, hydrogen peroxide, and short chain fatty acids, short chain fatty acid ester derivatives, or mixtures thereof (which may be derived from renewable sources) may provide cleaning and antimicrobial benefits at pHs ranging from about 1 to about 6.

### SUMMARY OF THE INVENTION

The present disclosure attempts to solve one or more of the needs above by providing a composition comprising from about 0.05% to about 40% of a surfactant comprising from about 6 to about 12 carbon atoms; from about 0.01% to about 40% of an acidifying agent; from about 0.01% to about 30% hydrogen peroxide; from about 0.01% to about 25% of a fatty acid of formula I, a fatty acid ester of formula I, or mixtures thereof:

R¹—C(O)O—R²

where R¹ is selected from the group consisting of linear or branched, substituted or unsubstituted C₆-C₁₀ hydrocarbyl groups and R² is selected from the group consisting of H and linear or branched, substituted or unsubstituted C₁-C₆ hydrocarbyl groups; from about 15% to about 99.95% water, where the composition has a pH of from about 1.0 to about 6.0.

### DETAILED DESCRIPTION OF THE INVENTION

Features and benefits of the disclosed invention will become apparent from the following description, which includes examples intended to give a broad representation of the invention. Various modifications will be apparent to those skilled in the art from this description and from practice of the invention. The scope is not intended to be limited to the particular forms disclosed and the invention covers all modifications, equivalents, and alternatives falling within the spirit and scope of the invention as defined by the claims.

As used herein, the articles including “the,” “a” and “an” when used in a claim or in the specification, are understood to mean one or more of what is claimed or described.

As used herein, the terms “include,” “includes” and “including” are meant to be non-limiting.

As used herein, the terms “active” and “agent” are used interchangeably.

As used herein, the term “renewable” (as in “renewable feedstock”) refers to materials (e.g., surfactant, solvent, acidifying agent) that are derived from a renewable feedstock and contain renewable carbon. The term “renewable” is used interchangeably with the terms “biobased” and “natural.” A renewable feedstock is a feedstock that is derived from a renewable resource, e.g., plants, and non-geologically derived. A material may be partially renewable (less than 100% renewable carbon content), 100% renewable (100% renewable carbon content), or somewhere in between (e.g., 50% renewable carbon content). A renewable material, for example a renewable ethanol, may be blended with a non-renewable material, for example, conventional ethanol, to yield a partially renewable material, e.g., partially renewable ethanol.

The terms “microorganism” or “microbe” as used herein are intended to include cellular organisms, both unicellular and multicellular that are less than 5 mm in length, and include but are not limited to bacteria, fungi, prions, enveloped and non-enveloped viruses, archaea, protists, protozoa or oocysts formed by protozoa, green algae, plankton, planarian, amoebas and yeasts, or spores formed by any of these. The terms “microorganism” or “microbe” include the single or planktonic microbes that may contaminate surfaces, as well as communities of microbes that grow as biofilms on surfaces.

The term “antimicrobial” as used herein refers to a compound that exhibits microbicide or microbiostatic properties that enables the compound to kill, destroy, inactivate, or neutralize a microorganism; or to prevent, mitigate, or reduce the growth, ability to survive, or propagation of a microorganism. In the context of antimicrobial, the term “treat” means to kill, destroy, inactivate, or neutralize a microorganism; or to prevent or reduce the growth, ability to survive, or propagation of a microorganism

The term “substantially free of” or “substantially free from” as used herein refers to either the complete absence of an ingredient or a minimal amount thereof merely as impurity or unintended byproduct of another ingredient. A composition that is “substantially free” of/from a component means that the composition comprises less than about 0.01%, or less than about 0.001%, or even 0%, by weight of the composition, of the component.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All cited patents and other documents are, in relevant part, incorporated by reference as if fully restated herein. The citation of any patent or other document is not an admission that the cited patent or other document is prior art with respect to the present invention.

In this description, all concentrations and ratios are on a weight basis of the antimicrobial composition unless otherwise specified.

### Composition

The compositions of the present disclosure may be formulated as concentrates or ready-to-use compositions. The compositions of the present disclosure may deliver cleaning benefits on hard and soft surfaces. The compositions of the present disclosure may be antimicrobial compositions and deliver improved antimicrobial activity on hard and soft surfaces. The compositions of the present disclosure may deliver cleaning benefits as well as antimicrobial benefits on hard and soft surfaces. The compositions disclosed herein may also be made in whole or in part using raw materials derived from renewable feedstocks, such as plant oils.

Concentrates may be diluted with water in order to provide an in-use solution having a desired level of detersive properties or other properties, including antimicrobial properties. The antimicrobial properties desired may depend on the challenge posed by the target microorganism; for example, enveloped viruses are more susceptible to inactivation than non-enveloped viruses, and spore-forming organisms are very resistant to chemical inactivation. Each organism type presents a different challenge and may call for a different level of dilution (or none) in order to achieve the desired antimicrobial activity.

The water used to dilute the concentrate (water of dilution) can be available at the locale or site of dilution. The water of dilution may contain varying levels of hardness depending upon the locale. Service waters available from various municipalities have varying levels of hardness. It is desirable to provide a concentrate that can handle the hardness levels found in the service water of various municipalities. The water of dilution may have a hardness ranging from about zero to at least about 400 ppm hardness (as CaCO₃).

Concentrated solutions may provide for improved economics to the manufacturer and to the user as they may comprise less water and may use less packaging material on a per-use basis, as compared to a ready-to-use composition. A concentrated composition may be diluted with water at a weight ratio of composition to water ranging from about 1:1.5 to about 1:1000, or from about 1:4 to about 1:250. The terms “in-use composition” or “in-use diluted composition” refer to concentrated compositions that have been diluted with water prior to use.

Alternatively, the compositions may be ready-to-use antimicrobial sprays, gels, creams, powders, soluble unit dose articles, pastes, or antimicrobial wet wipes. The compositions may also be comprised into foams, such as melamine-formaldehyde foams. For toilet hygiene, the compositions can be in the form of dissolvable rim-blocs which partially dissolve after the toilet is flushed. Ready to use compositions may provide more convenience to the user.

The compositions disclosed herein generally provide short-contact-time antimicrobial benefits, e.g., from about 10 seconds to about 3 minutes or from about 15 seconds to about 2 minutes, or from 30 seconds to about 1 minute, though longer contact times, e.g., from about 3 minutes to about 15 minutes, may also be achieved

Surfactant

The compositions of the present disclosure may comprise one or more surfactants. The surfactant may comprise from about 6 to about 12 carbon atoms, or from about 6 to about 11 carbon atoms, or from about 6 to about 10 carbon atoms, or from about 8 to about 10 carbon atoms. The surfactant may be branched or linear, saturated or unsaturated. The surfactant may be branched and comprise from about 6 to about 12 carbon atoms, or from about 6 to about 11 carbon atoms, or from about 6 to about 10 carbon atoms, or from about 8 to about 10 carbon atoms in the primary carbon chain, where “primary carbon chain” denotes the longest carbon-based chain that is uninterrupted by a heteroatom, such as O, S, N and P. For example, n-octyl sulfate has 8 carbon atoms in the primary carbon chain, 2-propyl-1-heptyl sulfate has 7 carbon atoms in the primary carbon chain, and dodecyl methyl ester sulfonate (C₁₀H₂₁—CH(SO₃⁻)—C(O)O—CH₃) has 11 carbon atoms in the primary carbon chain. In the context of branched surfactants, Cₙ (such as C₁ or C₈) refers to the number of carbon atoms in the primary carbon chain (for example, a 2-ethyl-1-hexyl primary carbon chain is C₆). In the context of linear (or unbranched) surfactants, Cₙ (such as C₁ or C₈) refers to the total number of carbon atoms in the surfactant. The surfactants may be substantially free of trace transition metal impurities.

The compositions may comprise from about 0.01% to about 60%, or from about 0.01% to about 40%, or from about 0.03% to about 35%, or from about 0.05% to about 30% of surfactant.

The concentrated compositions disclosed herein are generally intended to be diluted prior to use. A concentrated composition may comprise from about 0.5% to about 1%, or from about 1% to about 2%, or from about 2% to about 3%, or from about 3% to about 5%, or from about 5% to about 10%, or from about 10% to about 20%, or from about 20% to about 40%, of surfactant. A ready-to-use antimicrobial composition may comprise from about 0.01% to about 0.05%, or from 0.05% to about 0.10%, or from about 0.10% to about 0.15%, or from about 0.15% to about 0.25%, or from about 0.25% about 0.50% of surfactant. Ready-to-use antimicrobial compositions may comprise greater concentrations of surfactant, e.g., greater than about 0.50% (for example, for treatment of surfaces contaminated with mycobacteria, spore forming organisms, or biofilms).

Without being bound by theory, it is believed that the short chain-length of the surfactant—from about 6 to about 12 carbon atoms, or from about 6 to about 11 carbon atoms, or from about 6 to about 10 carbon atoms, or from about 8 to about 10 carbon atoms—is particularly beneficial for antimicrobial applications involving shorter contact times between the microorganism and the composition, for example, from about 10 seconds to about 3 minutes or from about 15 seconds to about 2 minutes, or from 30 seconds to about 1 minute. The short chain-length of the surfactant is believed to enhance the activity of antimicrobial active(s) in the composition. The short chain-length of the surfactant is also believed to help solubilize the (otherwise substantially water-insoluble) fatty acid or ester. Critical Micelle Concentration (CMC) measurements in the presence and absence of the fatty acid or ester indicate that the surfactants disclosed herein enhance the solubility of the fatty acid or ester by incorporating the fatty acid or ester into the micellar structure(s) of the surfactant. The CMC of the surfactant is significantly reduced, and this provides a reservoir of solubilized fatty acid or ester for antimicrobial potentiation activity. It is believed that the chain-length of the surfactant and the chain-length of the fatty acid or ester may be matched, for example, where the difference between the chain-length of the surfactant and the chain-length of the fatty acid or ester is about 2 to about 3 carbon atoms, to provide a combination of increased solubility of the fatty acid or ester in the composition and increased antimicrobial activity of the composition.

The compositions disclosed herein may comprise one or more C₁₃₋₁₈ surfactants. For example, commercial surfactants are generally made up of a blend of molecules having different alkyl chain lengths (though it is possible to obtain single chain-length cuts), e.g., Polystep® B-25 (from the Stepan Company) is described as sodium decyl sulfate but also contains about 25%-30% dodecyl sulfate, by weight of the alkyl sulfate surfactant. Similarly, many commercial lauryl surfactants may include about 30% or more surfactant having chain-length(s) greater than C₁₂. When C₁₃₋₁₈ surfactant is present in the composition(s), the weight ratio of C₆₋₁₂ surfactant to C₁₃₋₁₈ surfactant may be greater than about 2:1, or greater than about 3:1. The average chain-length of the surfactant in the composition(s) may be less than about C₁₂, or less than about C₁₁. The surfactant in the composition may have an average chain-length of from about C₇ to about C₈, or from about C₈ to about C₉, or from about C₉ to about C₁₀, or from about C₁₀ to about C₁₁. The composition(s) may comprise surfactant having an average chain-length of about C₈.

The solubility of the fatty acid or ester may be further increased by utilizing C₁₃₋₁₈ surfactants. However, increased surfactant chain-length further reduces CMC, which means that a reduced concentration of both surfactant monomer and fatty acid or fatty acid ester monomer is available for antimicrobial potentiation. Thus, there may be an optimal balance of shorter chain-length surfactant and longer chain-length surfactant, whereby the longer chain-length surfactant helps to solubilize the fatty acid or fatty acid ester via formation of mixed micelles, and the shorter chain-length surfactant increases the CMC of the surfactant and the concentration of surfactant monomers and fatty acid or fatty acid ester monomers that drive short-contact-time antimicrobial activity. The optimal balance may vary, depending on whether short-contact-time antimicrobial activity is desired. In other words, increased CMC values may be advantageous for faster activity, while reduced CMC values may be advantageous for longer-contact-time applications.

The antimicrobial compositions disclosed herein may comprise a surfactant comprising from about 6 to about 12 carbon atoms and a fatty acid or fatty acid ester of formula I (comprising from about 6 to about 12 carbon atoms). The CMC of the composition may be from about 100 ppm to about 2,500 ppm, or from about 200 ppm to about 2,000 ppm, or from about 300 ppm to about 1,500 ppm.

The composition(s) disclosed herein may comprise surfactant selected from the group consisting of an anionic surfactant, a cationic surfactant, a nonionic surfactant, an amphoteric surfactant, a zwitterionic surfactant, and mixtures thereof.

Suitable anionic surfactants include the sodium, potassium, ammonium, alkanol-ammonium magnesium and calcium salts of C₈-C₁₀ glyceryl ether sulfonates, C₈ alkyl sulfonates, C₂-C₈ linear alkyl benzene sulfonate, C₆-C₁₂ alkyl sulfates, C₈-C₁₂ alkyl ether sulfates, C₅₋₁₀ alkyl and alkenyl succinates as mono or dianionic surfactants [e.g., R—CH(COO⁻M⁺)-CH₂—COO⁻M⁺, R—CH(COO⁻M⁺)-CH₂—COOH⁻ and R—CH(COOH)—CH₂—COO⁻M⁺ wherein R=C₅₋₁₀ linear or branched alkyl or alkenyl group and M=lithium, sodium, potassium, ammonium or alkanol-ammonium, and mixtures thereof], C₈-C₁₂ methyl ester sulfonates, C₈-C₁₂ fatty acid sulfonates and C₆-C₁₂ carboxylates, and mixtures thereof. The surfactant may comprise an anionic surfactant selected from the group consisting of sodium octyl sulfate, sodium decyl sulfate, sodium octyl glyceryl ether sulfonate (C₈H₁₇—O—CH₂—CH(OH)—CH₂SO₃Na), the sodium salt of 2-propyl-1-heptyl sulfate, the sodium salts of C₉₋₁₁ secondary sulfates, the sodium salts of C₁₂ methyl ester sulfonate and C₁₂ fatty acid sulfonate, and mixtures thereof. The surfactant may comprise an anionic surfactant selected from the group consisting of octyl sulfate, sodium decyl sulfate, and mixtures thereof. The anionic surfactant may be derived from a renewable feedstock, for example, n-octyl alcohol or n-decyl alcohol derived from plant oils for the making of n-octyl sulfate and n-decyl sulfate, respectively, and n-dodecyl methyl ester derived from plant oils for the making of C₁₂ methyl ester sulfonate and C₁₂ fatty acid sulfonate.

Suitable nonionic surfactants include linear or branched, saturated or unsaturated alcohol alkoxylates, alkyl glycosides, and alkyl ethoxy carboxylic acids comprising from about 6 to about 12 carbon atoms in the primary chain. The surfactant may comprise a nonionic surfactant selected from the group consisting of C₆₋₁₂ alcohol ethoxylate comprising an average of from about 1 mole to about 7 moles of ethylene oxide, C₆₋₁₂ alcohol ethoxy propoxylate comprising an average of from about 1 mole to about 7 moles of ethylene oxide and from about 1 mole to about 4 moles of propylene oxide, C₈ pyrrolidone, C₈ and C₈₋₁₀ alkyl polyglucoside with a degree of glucoside polymerization of from about 1 to about 1.6, C₈₋₁₀ alkyl polypentoside (e.g., xyloside and riboside) with a degree of sugar pentoside polymerization of from about 1 to about 1.6, and C₁₂ ethoxy carboxylic acid comprising an average of from about 1 mole to about 3 moles of ethylene oxide. The surfactant may comprise a nonionic surfactant selected from the group consisting of octyl alkylpolyglycoside, decyl alkylpolyglycoside, octyl pyrrolidone, and mixtures thereof. As in the case of the anionic surfactant, the nonionic surfactant may be derived from a renewable feedstock. For example, the C₈ and C₈₋₁₀ alkyl polyglycosides (hexosides and pentosides) may be made from entirely renewable feedstocks.

Suitable cationic surfactants include saturated or unsaturated betaines, amine oxides, alkyl morpholinium compounds and alkyl trimethyl ammonium compounds comprising from about 6 to about 12 carbon atoms. The surfactant may comprise a cationic surfactant selected from the group consisting of n-octyl dimethyl amine oxide, n-octyl dimethyl betaine, n-octyl amidopropyl betaine, and mixtures thereof. The cationic surfactant may be derived from a renewable feedstock.

Suitable zwitterionic surfactants include 2-ethyl-1-hexyl imino dipropionate as well as n-dodecyl imino dipropionate (mono- and dianionic salts), C₆₋₁₂ amphoglycinates, and C₆₋₁₂ alkyl sulfobetaines, such as the sodium salt of n-octyl, n-decyl, or n-dodecyl N,N-dimethyl-3-ammonio-1-propanesulfonate.

The antimicrobial composition(s) disclosed herein may comprise surfactant selected from the group consisting of C₈ glyceryl ether sulfonate, C₆-C₁₂ alkyl sulfate, C₈-C₁₂ methyl ester sulfonate, C₈-C₁₂ fatty acid sulfonate, C₆-C₁₂ ether carboxylate, C₈₋₁₀ amine dimethyl oxide, C₈ pyrrolidone, C₈ dimethyl betaine, C₈₋₁₀ alkyl polyglycoside, C₈₋₁₂ N,N-dimethyl-3-ammonio-1-propanesulfonate, and mixtures thereof. The antimicrobial composition(s) disclosed herein may comprise from about 0.05% to about 30% of surfactant, where the surfactant is selected from the group consisting of sodium octyl sulfate, sodium decyl sulfate, sodium octyl glyceryl ether sulfonate, sodium dodecyl methyl ester sulfonate, sodium dodecyl fatty acid sulfonate, octyl dimethyl amine oxide, octyl pyrrolidone, and mixtures thereof.

Acidifying Agent

The compositions disclosed herein may comprise acidifying agent. The acidifying agent may adjust the pH of the composition to the following range: from about 1.0 to about 6.0, or from about 1.0 to about 5.5, or from about 1.0 to about 5.0, or from about 2.5 to about 5.0. The acidifying agent may help stabilize the pH of the composition by providing buffering capacity. The acidifying agent may also sequester transition metals, including iron, copper, manganese and the like. The acidifying agent may be chosen to further enhance the antimicrobial activity of the composition. The acidifying agent may be a US EPA/Health Canada registered active or a European notified antimicrobial substance.

The acidifying agent may comprise an organic acid, an inorganic acid, or a mixture thereof. The acidifying agent may be substantially free of trace transition metal impurities. Suitable inorganic acids include phosphoric acid, sulfuric acid, urea-sulfuric acid, hydrochloric acid, sulfamic acid, methyl sulfuric acid, hypochlorous acid, sodium bisulfate, and the like. Suitable organic acids include polymeric acids comprising at least 3 carboxylic acid groups, C₁-C₁₁ organic acids comprising at least one carboxylic acid group, and organic acids that do not comprise carboxylic acid functional groups (such as imidazole derivatives or phenolic or polyphenolic compounds). Non-limiting examples of polymeric acids include polymers of acrylic acid, methacrylic acid, maleic acid, or itaconic acid or copolymers of acrylic acid, methacrylic acid, maleic acid, itaconic acid, or mixtures thereof. Polymeric acids may be homopolymers or copolymers having a molecular weight of about 500 g/mol or greater. The polymeric acid may have a molecular weight ranging from about 500 g/mol to about 1,000,000 g/mol, or from about 500 g/mol to about 100,000 g/mol, or from about 1,000 g/mol to about 20,000 g/mol. Copolymers may be random copolymers or block copolymers. In addition to monomer units comprising carboxylic acid groups, the copolymers may also include one or more other monomers, such as styrene, acrylic ester, acrylamide, olefin sulfonate, and olefin acetate.

Non-limiting examples of C₁-C₁₁ organic acids include formic acid, acetic acid, benzoic acid, malonic acid, citric acid, maleic acid, fumaric acid, succinic acid, lactic acid, malic acid, tartaric acid, gluconic acid, glutaric acid, adipic acid, 2-ethyl-1-hexanoic acid, heptanoic acid, octanoic acid, nonanoic acid, undecylenic acid, butane tetracarboxylic acid, and the like. The organic acid may be derived from a renewable, plant-based feedstock and produced using natural processes, such as fermentation; examples include bio-based acetic acid, bio-based citric acid, bio-based lactic acid and bio-based succinic acid, and the like. The organic acid may have food-use pedigree or be Generally Regarded As Safe (GRAS) or a food additive by the US Food & Drug Administration.

The composition(s) disclosed herein may comprise acidifying agent, where the acidifying agent is selected from the group consisting of formic acid, acetic acid, benzoic acid, malonic acid, citric acid, maleic acid, fumaric acid, hypochlorous acid, succinic acid, gluconic acid, glutaric acid, lactic acid, 2-ethyl-1-hexanoic acid, octanoic acid, nonanoic acid, peracetic acid, peroctanoic acid, undecylenic acid, and mixtures thereof, or the acidifying agent is selected from the group consisting of benzoic acid, citric acid, lactic acid succinic acid, maleic acid, succinic acid, octanoic acid, and mixtures thereof.

The compositions may comprise from about 0.01% to about 40%, or from about 0.03% to about 25%, or from about 0.05% to about 10% acidifying agent. A concentrated composition may comprise from about 0.5% to about 1%, or from about 1% to about 3%, or from about 3% to about 5%, or from about 5% to about 10%, or from about 10% to about 20%, or from about 20% to about 40% of acidifying agent. An increased concentration of acidifying agent increases the composition's reserve buffering capacity, which reduces pH fluctuation upon dilution. Partial neutralization of the acidifying agent to a pH value just below its pKa (e.g., 0.1 to 0.5 pH units below the acidifying agent's pKa) may also help to reduce pH fluctuation upon dilution. A concentrate may therefore be formulated at an increased pH, which may lead to an improved overall safety profile without compromising in-use (diluted) antimicrobial performance. A ready-to-use composition may comprise from about 0.01% to about 0.05%, or from about 0.05% to about 0.10%, or from about 0.10% to about 0.15%, or from about 0.15% to about 0.25%, or from about 0.25% to about 0.50% of acidifying agent. Ready-to-use compositions may comprise greater amounts of acidifying agent, e.g., greater than 0.50%, for example, to treat surfaces contaminated with mycobacteria, spore forming organisms, or biofilms.

Generally, an increased pH may improve the overall safety of the composition, enhance the compatibility of the composition with a larger variety of optional adjuncts, and increase the scope of applications for which the composition may be used. For compositions having pH 3.5 or greater, the acidifying agent may be selected from acidifying agents having pKa values greater than about 4.0; non-limiting examples of such acidifying agents include acetic acid (pKa=4.8), succinic acid (pKa 4.2), benzoic acid (pKa=4.2), trans-cinnamic acid (pKa=4.4), p-coumaric acid (4-hydroxy cinnamic acid, pKa=4.6), octanoic acid (pKa 4.9), undecylenic acid (pKa 5.0), heptanoic acid (pKa=5.1), nonanoic acid (pKa=5.2), imidazole (pKa=7.0), hypochlorous acid (pKa=7.0) and mixtures thereof. Diprotic acid salts, such as the monosodium salt of maleic acid (pKa2=6.1), and triprotic acid salts, such as the mono- and dibasic salts of citric acid (pKa2=4.5, pKa3=6.4), may also be used to adjust the pH of the composition to pH 4.0 and greater.

The weight ratio of surfactant to acidifying agent in the composition may be from about 50:1 to about 1:50, or from about 10:1 to about 1:10, or from about 5:1 to about 1:5, or from about 3:1 to about 1:3.

The acidifying agent may be chosen to potentiate or provide antimicrobial properties. The acidifying agent may be selected from the group consisting of benzoic acid, citric acid, succinic acid, glycolic acid, lactic acid, octanoic acid, hypochlorous acid, peroxyacetic acid, peroxyoctanoic acid, and mixtures thereof. Acids characterized by reduced water solubility, including succinic acid, benzoic acid, cinnamic acid and octanoic acid, may be especially beneficial.

The acidifying agent is preferably not a C₆₋₁₀ fatty acid.

Hydrogen Peroxide

The composition may comprise from about 0.01%, or from about 0.03%, or from about 0.05%, or from about 0.06%, or from about 0.07% to about 8%, or to about 10%, or to about 15%, or to about 20%, or to about 30% hydrogen peroxide. The composition may comprise from about 0.01%, or from about 0.03%, or from about 0.05%, or from about 0.06%, or from about 0.07% to about 8%, or to about 10%, or to about 15%, or to about 20%, or to about 30% of hydrogen peroxide by weight of the composition. The concentration of hydrogen peroxide in the composition depends on the desired concentration of the overall composition (e.g., concentrate versus ready-to-use) as well as on the antimicrobial benefits sought.

Compositions comprising hydrogen peroxide may comprise less than about 5 ppm transition metal ion impurities, or less than about 2 ppm transition metal ion impurities, or less than 0.5 ppm transition metal ion impurities. Compositions comprising hydrogen peroxide may comprise less than about 5 ppm ferrous ion, less than about 5 ppm ferric ion, or less than about 5 ppm of a mixture thereof, or less than about 1 ppm ferrous ion, less than about 1 ppm ferric ion, or less than about 1 ppm of a mixture thereof, or less than about 0.1 ppm ferrous ion, less than about 0.1 ppm ferric ion, or less than about 0.1 ppm of a mixture thereof.

For concentrates, the concentration of hydrogen peroxide may be from about 1% to about 30%, or from about 1% to about 15%, or from about 1% to about 8%, or from about 1% to about 5%, or from about 1% to about 3%, or from about 3% to about 30%, or from about 3% to about 15%, or from about 3% to about 8%, or from about 3% to about 5%, or from about 5% to about 30%, or from about 5% to about 15%, or from about 5% to about 8%, or from about 8% to about 30%, or from about 8% to about 15%, or from about 15% to about 30% by weight of the composition. For ready-to-use compositions or in-use diluted compositions, the concentration of hydrogen peroxide may be from about 0.02% to about 1%, or from about 0.02% to about 0.50%, or from about 0.02% to about 0.25%, or from about 0.02% to about 0.15%, or from about 0.02% to about 0.10%, or from about 0.02% to about 0.05%, or from 0.05% to about 1%, or from about 0.05% to about 0.50%, or from about 0.05% to about 0.25%, or from about 0.05% to about 0.15%, or from about 0.05% to about 0.10%, or from 0.10% to about 1%, or from about 0.10% to about 0.50%, or from about 0.10% to about 0.25%, or from about 0.10% to about 0.15%, or from about 0.15% to about 1%, or from about 0.15% to about 0.50%, or from about 0.15% to about 0.25%, or from about 0.25% to about 1%, or from about 0.25% to about 0.50%, or from about 0.50% to about 1% by weight of the composition. A ready-to-use composition may comprise a greater concentration of hydrogen peroxide, e.g., greater than about 1%, for example, for challenging antimicrobial benefits, such as the treatment of surfaces contaminated with mycobacteria, spore forming organisms, or biofilms.

The concentration of hydrogen peroxide may be from about 0.01% to about 8.0%. The weight ratio of hydrogen peroxide to acidifying agent is from about 0.1:1 to about 10:1, or from about 0.2:1 to about 5:1, or from about 0.5:1 to about 2:1.

For compositions comprising hydrogen peroxide, as the optional antimicrobial active, the weight ratio of surfactant to hydrogen peroxide may be from about 0.05:1 to about 20:1, or from about 0.1:1 to about 10:1, or from about 0.2:1 to about 5:1.

Hydrogen peroxide is a registered antimicrobial active ingredient in the US and Canada, and it is a notified substance in Europe. The compositions disclosed herein may comprise hydrogen peroxide in combination with an unregistered (North America) or unnotified (Europe) acidifying agent. The compositions disclosed herein may comprise hydrogen peroxide in combination with a registered (North America) or notified (Europe) acidifying agent. The compositions disclosed herein may comprise benzoic acid, citric acid, glycolic acid, hypochlorous acid, lactic acid, octanoic acid, peroxyacetic acid, hydrogen peroxide, ionic silver, or mixtures thereof (which are US EPA and Health Canada registered antimicrobial actives). Benzoic acid, citric acid, lactic acid, hydrogen peroxide, and certain ionic silver compounds, such as silver nitrate, are also approved for use for water treatment or on food contact surfaces in the USA. Additionally, citric acid, 1-lactic acid, ethanol, isopropanol, sodium bisulfate and hydrogen peroxide are the only antimicrobial approved actives for the US EPA's Design for the Environment (DfE) pesticide pilot project. Lactic acid, citric acid, peroxyoctanoic acid, and hydrogen peroxide are also notified substances in the European Union. These certifications may provide important credentialing options for the compositions disclosed herein.

The compositions disclosed herein may comprise hydrogen peroxide and peracid, where the peracid is formed in-situ via the reaction of a carboxylic acid-containing acidifying agent and hydrogen peroxide. For example, when the composition comprises octanoic acid or nonanoic acid, there may be equilibrium concentrations of peroxyoctanoic acid or peroxynonanoic acid, respectively, formed in-situ in the composition. The rate of formation of the peracid may depend on the pH of the composition (reduced pHs favor peracid formation and faster rates of formation).

C₆₋₁₀ Fatty Acid or C₆₋₁₀ Fatty Acid Ester

The compositions disclosed herein may comprise a fatty acid or fatty acid ester of formula I:

R¹—C(O)O—R²

where R¹ is selected from the group consisting of linear or branched, substituted or unsubstituted C₆-C₁₀ hydrocarbyl groups and R² is selected from the group consisting of H and linear or branched, substituted or unsubstituted C₁-C₆ hydrocarbyl groups. R¹ may be selected from the group consisting of —O₅H₁₁, —C₆H₁₃, —C₇H₁₅, —C₈H₁₇, and —C₉H₁₉. The compositions disclosed herein may comprise a mixture of two or more different fatty acids or a mixture of fatty acid(s) and ester(s). The composition may comprise a fatty acid selected from the group consisting of heptanoic acid, octanoic acid, nonanoic acid, decanoic acid, and mixtures thereof. The composition may comprise octanoic acid, nonanoic acid and mixtures thereof. The composition may comprise a fatty acid ester selected from the group consisting of the methyl, ethyl, isopropyl, propylene glycol, dipropylene glycol, glycerol, and diglycerol esters of hexanoic acid, the methyl, ethyl, isopropyl, propylene glycol, dipropylene glycol, glycerol, and diglycerol esters of octanoic acid, the methyl, ethyl, isopropyl, propylene glycol, dipropylene glycol, glycerol, and diglycerol esters of nonanoic acid, the methyl, ethyl, isopropyl, propylene glycol, dipropylene glycol, glycerol, and diglycerol esters of decanoic acid, and mixtures thereof. The composition may comprise a fatty acid ester selected from the group consisting of the methyl and ethyl esters of hexanoic acid, the methyl, ethyl, and monoglycerol esters of octanoic acid, the monoglycerol ester of decanoic acid, and mixtures thereof. The composition may comprise a fatty acid ester selected from the group consisting of the monoglycerol ester of octanoic acid, the monoglycerol ester of decanoic acid, and mixtures thereof. The monoglycerol ester of octanoic acid and the monoglycerol ester of decanoic acid are known food grade emulsifiers.

The compositions disclosed herein may comprise from about 0.01%, or from about 0.03%, or from about 0.05% to about 15%, or to about 20%, or to about 25% by weight of fatty acid of formula I, fatty acid ester of formula I, or mixtures thereof.

A concentrated composition may comprise from about 0.5% to about 25%, or from about 0.5% to about 8%, or from about 0.5% to about 4%, or from about 0.5% to about 2%, or from about 0.5% to about 1%, by weight of fatty acid of formula I, fatty acid ester of formula I, or mixtures thereof. A concentrated composition may comprise from about 1% to about 25%, or from about 1% to about 8%, from about 1% to about 4%, or from about 1% to about 2% by weight of fatty acid of formula I, fatty acid ester of formula I, or mixtures thereof. A concentrated composition may comprise from about 2% to about 25%, or from about 2% to about 8%, or from about 2% to about 4% by weight of fatty acid of formula I, fatty acid ester of formula I, or mixtures thereof. A concentrated composition may comprise from about 4% to about 25%, or from about 4% to about 8% by weight of fatty acid of formula I, fatty acid ester of formula I, or mixtures thereof. A concentrated composition may comprise from about 8% to about 25% by weight of fatty acid of formula I, fatty acid ester of formula I, or mixtures thereof.

A ready-to-use composition may comprise from about 0.01% to about 0.50%, or from about 0.01% to about 0.20%, or from about 0.01% to about 0.10%, or from about 0.01% to about 0.05% by weight of fatty acid of formula I, fatty acid ester of formula I, or mixtures thereof. A ready-to-use composition may comprise from about 0.05% to about 0.50%, or from about 0.05% to about 0.20%, or from about 0.05% to about 0.10% by weight of fatty acid of formula I, fatty acid ester of formula I, or mixtures thereof. A ready-to-use composition may comprise from about 0.10% to about 0.50%, or from about 0.10% to about 0.20% by weight of fatty acid of formula I, fatty acid ester of formula I, or mixtures thereof. A ready-to-use composition may comprise from about 0.20% to about 0.50% by weight of fatty acid of formula I, fatty acid ester of formula I, or mixtures thereof. Ready-to-use compositions may comprise greater than 0.50% by weight of fatty acid of formula I, fatty acid ester of formula I, or mixtures thereof, for example, to treat surfaces contaminated with mycobacteria, spore forming organisms, or biofilms.

The weight ratio of surfactant to fatty acid of formula I, fatty acid ester of formula I, or mixtures thereof may be from about 0.1:1 to about 20:1, or from about 0.25:1 to about 10:1, or from about 0.5:1 to about 5:1. The weight ratio of surfactant to fatty acid of formula I, when present in the composition, may be from about 0.5:1 to about 10:1, or from about 1:1 to about 8:1, or from about 2:1 to about 5:1. The weight ratio of surfactant to fatty acid ester of formula I, when present in the composition, may be from about 0.25:1 to about 10:1, or from about 0.5:1 to about 5:1, or from about 0.75:1 to about 3:1.

The fatty acids disclosed herein are widely available from chemical suppliers. For example, hexanoic acid, octanoic acid, and decanoic acid are available in a variety of grades from The Procter & Gamble Company. A mixture of methyl octanoate and methyl decanoate is available as ME-810® from The Procter & Gamble Company. Abitec Corporation supplies propylene glycol monooctanoate (also known as propylene glycol monocaprylate) under the tradename Capmul® 908P, glycerol monooctanoate (also known as glyceryl caprylate) under the tradename Capmul® 708G, and glycerol monodecanoate (also known as glyceryl monocaprate) under the tradename Capmul® 9010G. The fatty acid or fatty acid ester of formula I may be derived from renewable sources, such as plant oils. Also, the glycerol raw material used in producing the glycerol esters disclosed herein may be obtained from renewable sources.

It is understood that fatty acids and fatty acid esters are typically sold as mixtures of several different compounds, rather than as a single, pure compound. For example, Capmul® 708G, the monoester of octanoic acid, contains a mixture of chemical compounds—the monoester accounts for about 80%-90% of the raw material ester content and the diester accounts for most of the remainder of the raw material ester content. Thus, the compositions disclosed herein may comprise diesters, especially as byproducts of the processes used to produce monoester raw materials.

It is believed that the compositions disclosed herein provide antimicrobial activity against a variety of microorganisms, including Gram-positive bacteria, Gram-negative bacteria, non-enveloped viruses, fungi, mycobacteria, and even spore-forming organisms, such as Clostridium difficile spores. Without wishing to be bound by theory, it is believed that the lipophilic character of the fatty acid of formula I or the fatty acid ester of formula I contributes to this antimicrobial activity; the fatty acid or fatty acid ester is believed to preferentially partition into the microorganism, versus remaining in its monomer form in the composition. This partitioning is believed to induce micelles in the composition to release more fatty acid and/or fatty acid ester monomers, in order to re-establish thermodynamic equilibrium. The released fatty acid and/or fatty acid ester monomers again preferentially partition into the microorganism and the whole series of events—where fatty acid and/or fatty acid ester monomers are continuously created from micelles and are then used up against the target microorganism—may contribute to the rapid antimicrobial activity of the disclosed compositions. By quickly and continuously permeating though microorganism defenses, the fatty acid and/or fatty acid ester may also potentiate the activity of antimicrobial actives (e.g., hydrogen peroxide) that are present in the composition.

Also, compositions that comprise hydrogen peroxide may exhibit enhanced Fenton chemistry, with iron or copper from the microorganism, and may generate increased concentrations of oxygen-based radicals, which may react with the fatty acid and/or fatty acid ester to form peracids or other highly reactive oxygen species (particularly, but not necessarily, inside the microorganism).

The fatty acid ester(s) disclosed herein may hydrolyze, over time, to its corresponding fatty acid, due to the acidic pH of the composition, particularly at a pH ranging from about 1.0 to about 3.0, and/or at increased temperatures (e.g., above room temperature). The fatty acid formed by ester hydrolysis is also believed to be solubilized by the C₆-C₁₂ surfactant(s) disclosed herein. In cases where hydrolysis may occur, the inclusion of ester in the composition may provide a reservoir of fatty acid. Also, fatty acid esters typically have desirable odor profiles.

The concentration of fatty acid formed by ester hydrolysis may optionally be adjusted by the addition of from about 0.1% to about 10% of a lower alcohol. Suitable lower alcohols include methanol, ethanol, propylene glycol, dipropylene glycol, diethylene glycol, glycerol, diglycerol, polyglycerol, or C₁ to C₈ mono- or di-glycerol ethers. The compositions disclosed herein may comprise glycerol and/or glycerol derivatives, particularly when acid (re)esterification is sought. The compositions disclosed herein may comprise one or more C₆₋₁₀ fatty acid(s) of formula I and from about 1% to about 10% by weight glycerol, which may increase the concentration(s) of C₆₋₁₀ glycerol mono-, di-, and triesters in the composition. An increase in the concentration of fatty acid esters may also provide a desirable odor profile. The compositions disclosed herein may comprise one or more C₆₋₁₀ fatty acid ester(s) of formula I, from about 1% to about 10% by weight glycerol, and be substantially free of C₆₋₁₀ fatty acid. The compositions disclosed herein may be substantially free of fatty acids and peracids, formed via hydrolysis and perhydrolysis, respectively, of the esters disclosed herein. For compositions comprising an ester of formula I and hydrogen peroxide, peracids may be formed via the following reactions:

R¹—C(O)O(R²)+H₂O=R¹C(O)OH+R²OH; and

R—C(O)OH+H₂O₂=R—C(O)OOH+H₂O.

As ester hydrolysis is catalyzed by acid, increasing the pH of the composition may reduce ester hydrolysis, thereby reducing the concentration of peracid.

The weight ratio of C₆₋₁₀ fatty acid to corresponding C₆₋₁₀ fatty peracid may be from about 5:1 to about 1000:1, or from about 10:1 to about 500:1, or from about 15:1 to about 100:1. The compositions may comprise a combination of C₆₋₁₀ peracid and a shorter-chain (C₁—O₅) peracid (e.g., peracetic acid).

The composition may further comprise one or more esters of formic acid, acetic acid, benzoic acid, lactic acid, succinic acid, 3-hydroxybutyric acid and citric acid, such as isobutyl formate, butyl acetate, ethyl benzoate, ethyl lactate, butyl 3-hydroxybutyrate, or triethyl citrate. The compositions may further comprise peracid from the in-situ reaction of acidifying agent with hydrogen peroxide or peracid formed by hydrolysis/perhydrolysis of the esters in the composition; alternatively, the compositions may be substantially free of peracid, especially peracid formed by hydrolysis/perhydrolysis of the esters in the compositions.

The concentration of in-situ generated C₆₋₁₀ peracid in the composition may be from about 0 ppm, or from about 0.5 ppm, or from about 1 ppm, to about 10 ppm, or to about 15 ppm, or to about 25 ppm, or to about 50 ppm. Surprisingly, the disclosed combination of C₆-C₁₂ surfactant, acidifying agent, and hydrogen peroxide, in combination with a low concentration of in-situ generated C₆₋₁₀ peracid, provides bactericidal activity at short exposure times (e.g., 15 seconds to 2 minutes).

Water

The compositions disclosed herein may comprise water. The water may be of any hardness. The water may be de-ionized water, reverse-osmosis-treated water, distilled water, or soft water (typically, soft water does not exceed 40 ppm hardness (as CaCO₃)). The water may be de-ionized and/or reverse osmosis treated and may comprise less than about 1 ppm transition metal ion, or less than about 100 ppb transition metal ion.

The compositions may comprise from about 15% to about 99.95%, or from about 20% to about 95%, or from about 20% to about 90%, or from about 25% to about 85% water by weight of the composition. The amount of water in a given composition depends on the degree to which the composition is concentrated. A super concentrate composition may comprise less than about 50%, or from about 15% to about 40%, or from about 20% to 35% of water by weight of the composition. Such super concentrates may provide improved economics on a per-use basis (e.g., following recommended dilution) for the user. Also, the water activity of a super concentrate may be sufficiently reduced, such that the composition does not freeze at temperatures as low as −3° C., or as low as −18° C. Super concentrates may also exhibit improved ambient-temperature (e.g., 20-23° C.) phase stability. Compositions comprising increased water content may also freeze more readily and exhibit phase instability upon thawing (e.g., crystallization or precipitation of one or more components). Super concentrate compositions may comprise surfactant, for example, a surfactant comprising from about 10 to about 12 carbon atoms; the surfactant may improve the ambient-temperature phase stability of the super concentrate compositions, upon dilution with water.

Ready-to-use compositions generally comprise greater water than concentrated compositions, which are intended to be diluted at the point of use. A ready-to-use composition may comprise from about 70% to about 99.9%, or from about 75% to about 99.5% water, or from about 80% to about 99% water by weight of the composition.

pH

The compositions disclosed herein may have pHs ranging from about 1.0 to about 6.0, or from about 1.0 to about 5.5, or from about 1.0 to about 5.0, or from about 2.5 to about 6.0, or from about 2.5 to about 5.5, or from about 2.5 to about 5.0, or from about 3.0 to about 6.0, or from about 3.5 to about 5.5, or from about 4.0 to about 5.5. For a concentrated composition that comprises less than about 70% water, the pH is measured after adding de-ionized water to the composition, until the total concentration of water in the composition is about 70%. For compositions that comprise greater than or equal to about 70% water, pH is measured on the composition as made (the composition is not diluted prior to measuring the pH).

Adjuncts

The compositions disclosed herein may also contain one or more adjuncts. Adjuncts may be employed to increase immediate and/or residual efficacy of the compositions, improve the wetting characteristics of the compositions upon application to a target substrate, operate as solvents for diluted compositions, and/or serve to modify the aesthetic characteristics of the composition. These adjuncts may also provide degreasing and solubilizing benefits, additional antimicrobial potentiation, thickening, soil agglomeration or soil release benefits, enhanced composition solubility, further catalysis of antimicrobial activity, residual or long-lasting (e.g., 24 hours) duration antimicrobial properties and/or enhanced surface safety benefits.

The composition(s) disclosed herein may comprise an adjunct selected from the group consisting of chelants, builders, buffers, abrasives, electrolytes, bleaching agents, fragrances, dyes, foaming control agents, corrosion inhibitors, essential oils, thickeners, pigments, gloss enhancers, enzymes, detergents, solvents, dispersants, polymers, silicones, hydrotropes, and mixtures thereof.

The compositions disclosed herein may further comprise an amide of formula II:

R³—CO—NR⁴R⁵ (II)

where R³ is selected from the group consisting of linear or branched, substituted or unsubstituted C₆-C₁₂ hydrocarbyl groups, each of R⁴ and R⁵ is independently selected from the group consisting of H, OH, a halogen group, and linear or branched, substituted or unsubstituted C₁-C₆ hydrocarbyl groups.

### Solvents

The composition(s) disclosed herein may comprise a solvent. Solvents are generally liquid at ambient temperature conditions. Solvents may be desirable adjuncts especially for ready-to-use compositions and concentrated compositions, which are diluted with water at a ratio of about 1 part concentrate to less than about 10 parts water or about 1 part concentrate to less than about 5 parts water. The compositions disclosed herein may comprise from about 0.25% to about 25%, or from about 0.5% to about 15%, or from about 1% to about 10% of solvent by weight of the composition. Solvents may be used to control suds, adjust composition viscosity, or provide additional antimicrobial potentiation. Solvents may also be used to improve cleaning or prevent components of the composition from crystallizing out. Non-limiting examples of solvents that may improve cleaning include glycol ethers, more specifically C₁-C₈ derivatives of mono-, di-, and triethylene glycol ethers and diethers, and the C₁-C₆ derivatives of mono-, di- and tripropylene glycol ethers and diethers. Non-limiting examples include propylene glycol propyl ether, dipropylene glycol butyl ether, diethylene glycol butyl ether, tripropylene glycol dimethyl ether, ethylene glycol n-hexyl ether, ethylene glycol n-octyl ether, and the like. “Butyl” includes normal butyl, isobutyl and tertiary butyl groups. The solvent may be chosen to be non-VOC (Volatile Organic Compound), as defined by the California Air Resources Board, or VOC, e.g., ethanol, isopropanol and propylene glycol. A VOC solvent may be present at a concentration of less than about 0.5% by weight of the in-use composition.

The composition(s) disclosed herein may comprise a solvent selected from the group consisting of ethanol, isopropanol, C₁-C₈ monoethylene glycol ether, C₁-C₈ diethylene glycol ether, C₁-C₈ triethylene glycol ether, C₁-C₆ monopropylene glycol ether, C₁-C₆ dipropylene glycol ether, C₁-C₆ tripropylene glycol ether, C₁-C₆ esters of formic acid, C₁-C₆ esters of acetic acid, C₁-C₆ esters of benzoic acid, C₁-C₆ esters of lactic acid, C₁-C₆ esters of 3-hydroxybutyric acid, C₁-C₆ amines, C₁-C₆ alkanol amines, and mixtures thereof. Examples of commercially available ethylene glycol-based solvents include Hexyl Cellosolve™ (ethylene glycol n-hexyl ether, C6 monoethylene glycol) and Butyl Carbitol™ (diethylene glycol n-butyl ether, C4 diethylene glycol) sold by Dow Chemical Company. Examples of commercially available propylene glycol-based solvents include Dowanol DPnB™ (dipropylene glycol n-butyl ether, C4 dipropylene glycol) and Dowanol TPM™ (tripropylene glycol methyl ether, C1 tripropylene glycol), which are also available from the Dow Chemical Company.

The composition(s) disclosed herein may comprise from about 1% to about 10% of a solvent selected from the group consisting of glycerol, diethylene glycol monoethyl ether, butyl 3-hydroxybutyrate, and mixtures thereof. Incorporation of diethylene glycol monoethyl ether, a non-VOC compound, may help solubilize highly crystalline and substantially water-insoluble materials, such as benzoic acid (acidifying agent) and octanohydroxamic acid (amide of formula II). Incorporation of diethylene glycol monoethyl ether may also improve freeze-thaw stability of the composition, particularly for compositions comprising benzoic acid, and/or octanohydroxamic acid, which are substantially water-insoluble, highly crystalline materials that may precipitate out or crystallize out when a composition is cooled during the freeze process or warmed during the thaw process.

For composition(s) having a pH of about 2.5 or greater, or a pH of about 3.0 or greater, an ester-based solvent may improve cleaning. Non-limiting examples of ester-based solvents include C₁-C₆ esters of formic acid, C₁-C₆ esters of acetic acid, C₁-C₆ esters of lactic acid, C₁-C₆ esters of citric acid, C₁-C₆ esters of succinic acid, and C₁-C₆ esters of 3-hydroxybutyric acid. The composition may comprise butyl 3-hydroxybutyrate (Omnia™ solvent, available from Eastman), which may provide a boost in cleaning performance, especially for greasy soils. Butyl 3-hydroxybutyrate may also help solubilize highly crystalline and substantially water-insoluble materials and promotes the freeze-thaw stability of the composition (particularly a composition comprising benzoic acid and/or octanohydroxamic acid). Other ester-based solvents of interest include derivatives of furan including but not limited to methyl-2-furoate, dimethyl-2,5-difuroate, 5-methyl-2-methyl furoate, Examples of furan-based solvents are available from xF technologies under the tradename ‘408’.

Ready-to-use compositions comprising benzoic acid, octanohydroxamic acid, or mixtures thereof, may comprise greater than about 0.5%, or greater than about 1%, by weight of the composition, of ester-based solvent, such as butyl 3-hydroxybutyrate. At such concentrations, butyl 3-hydroxybutyrate may mitigate or prevent crystallization. Concentrated compositions may comprise from about 3% to about 10% of ester-based solvent, such that upon dilution, the in-use level is at least about 0.5%, or at least about 1% by weight of the in-use composition.

Antimicrobial Active

The compositions herein may comprise an antimicrobial active in addition to hydrogen peroxide. The antimicrobial active is a material recognized by a governmental agency to provide antimicrobial activity. The antimicrobial active may be selected from the group consisting of benzoic acid, citric acid, glycolic acid, lactic acid, octanoic acid, nonanoic acid, decanoic acid, hypochlorous acid, peroxyacetic acid, peroxyoctanoic acid, ionic silver compounds, and mixtures thereof. The compositions disclosed herein may also optionally comprise from about 0.1% to about 10%, or from about 0.1% to about 5%, or from about 0.1% to about 1%, of a cationic antimicrobial agent. Suitable cationic antimicrobial agents for use in the compositions disclosed herein include benzalkonium chloride, benzethonium chloride, chlorhexidine diacetate, polyhexamethylene biguanide (PHMB), chlorhexidine digluconate, and mixtures thereof. The compositions may also comprise an antimicrobial agent selected from the group consisting of glutaraldehyde, zinc 2-pyridinethiol-1-oxide, copper sulfate pentahydrate, iodine, iodine salts, butoxypolypropoxypolyethoxyethanol iodine complex, polyvinylpyrrolidone-iodine complex, and mixtures thereof.

The additional antimicrobial active may be ionic silver. As used herein, “ionic silver,” refers to any silver (I) compound that may be solubilized or dispersed in an aqueous medium at a pH ranging from about 1.0 to about 6.0. Examples of ionic silver include silver acetate, silver lactate, silver nitrate, silver dihydrogen citrate, silver sulfate, silver citrate, as well as complexes of silver I formed with ammonia. The composition may comprise from about 0.001%, or from about 0.002%, or from about 0.003%, or from about 0.005% to about 0.25%, or to about 0.3%, or to about 0.5%, or to about 2% of ionic silver by weight of the composition.

The concentration of ionic silver is calculated as the weight percent of silver present in an ionic silver compound. For example, the weight percent of ionic silver in a composition comprising 0.1% silver nitrate is 0.064% [0.1%*(107.9/169.9)] and the weight percent of silver in a composition comprising 0.1% silver dihydrogen citrate is 0.036% [0.1%*107.9/300.0]. The concentration of ionic silver in the composition depends on the desired concentration of the overall composition (e.g., concentrate versus ready-to-use) as well as the antimicrobial benefits sought. Compositions comprising ionic silver may be substantially free of chloride ion, iodide ion, and/or bromide ion impurities; the compositions may comprise less than about 10 ppm chloride ion, less than about 10 ppm iodide ion, less than about 10 ppm bromide ion, or less than about 10 ppm of a mixture thereof, or less than about 1 ppm chloride ion, less than about 1 ppm iodide ion, less than about 1 ppm bromide ion, or less than about 1 ppm of a mixture thereof.

Essential Oils

Suitable essential oils or actives thereof include those essential oils which exhibit antimicrobial activity. By “actives of essential oils” it is meant any ingredient of essential oils that exhibits antimicrobial activity. Essential oils and actives thereof may also provide a desirable odor profile.

Suitable essential oils include, but are not limited to, those obtained from thyme, lemongrass, citrus, lemons, oranges, anise, clove, aniseed, cinnamon, geranium, roses, mint, lavender, citronella, eucalyptus, peppermint, camphor, sandalwood, cedar, or mixtures thereof.

Actives of essential oils include, but are not limited to, thymol (present, for example, in thyme), eugenol (present, for example, in cinnamon and clove), menthol (present, for example, in mint), geraniol (present, for example, in geranium and rose), verbenone (present, for example, in vervain), eucalyptol and pinocarvone (present in eucalyptus), cedrol (present, for example, in cedar), anethol (present, for example, in anise), carvacrol, hinokitiol, berberine, terpineol, limonene, or mixtures thereof. The compositions disclosed herein may comprise thymol. Thymol is commercially available, for example, from Sigma Aldrich.

Chelants

The compositions disclosed herein may comprise one or more chelants or sequestrants. As used herein, the terms “chelant” and “sequestrant” are used interchangeably. Chelants include chemical compounds that sequester alkali earth metal divalent ions, transition metal divalent ions, and/or transition metal trivalent ions from solution. The metal ions to be sequestered may be present in the compositions disclosed herein (for example, incorporated via hard water used in a dilution) or may be embedded within the microorganism that the composition is intended to treat. Metal ions in the concentrated compositions disclosed herein may originate from impurities in the water or in the raw materials used to make the compositions. These metal ions may adversely affect performance or composition stability. The concentration of metal ions may be reduced using processes to purify water, including reverse osmosis and de-ionization. Examples of such metal ions include the divalent and trivalent ions of iron, nickel, manganese, and the like.

Metal ions associated with a microorganism may be important for the functioning and survival of microorganism. The metal ions may be extracellular or they may be intracellular; the metal ions may be present, for example, at the active site of metabolic or regulatory enzymes or as a cofactor that enables enzymatic activity. Examples of metal ions associated with a microorganism include iron, copper, zinc and magnesium ions, and the like.

Highly water-soluble chelants may be used to sequester metal ions present in the composition. Lipophilic chelants may be used to target the metal ions associated with a microorganism. The composition(s) disclosed herein may comprise a mixture of hydrophilic and lipophilic chelants.

The composition(s) disclosed herein may comprise up to about 10%, by weight of the composition, or from about 1% to about 10% of chelant. The chelant may comprise one or more phosphorus atoms. Non-limiting examples of phosphorous-containing chelants include 1-hydroxyethylidene-1,1-diphosphonic acid (HEDP, also known as etidronic acid), diethylene triamine penta(methylene phosphonic acid), 2-phosphonobutane-1,2,4-tricarboxylic acid (PBTC), and the like. The chelant may be a nil-phosphorus chelant. Non-limiting examples of nil-phosphorus chelants include the sodium, potassium, and alkanolamine salts of nitrilotriacetic acid (NTA), methyl glycine diacetic acid (MGDA), glutamic N.N-diacetic acid (GLDA), ethylene diamine tetraacetic acid (EDTA), diethylene triamine pentaacetic acid (DTPA), iminodisuccinic acid (IDS), ethylenediamine N,N′-disuccinic acid (EDDS), 4,5-dihydroxy-1,3-benzene disulfonic acid (Tiron), 2-hydroxypyridine N-oxide (HPNO), octyl isothiazolinone (OIT), picolinic acid, dipicolinic acid, 1-hydroxy-4-methyl-6-(2,4,4-trimethylpentyl) pyridine-2-one (piroctone acid), and the like. 2-hydroxypyridine N-oxide (HPNO) may be employed for chelation as well as for reducing precipitation or crystallization of crystalline components, such as benzoic acid or octanohydroxamic acid.

The composition(s) disclosed herein may comprise from about 1% to about 10% of a chelant selected from the group consisting of diethylene triamine pentaacetic acid (DTPA), iminodisuccinic acid, ethylenediamine N,N′-disuccinic acid, 4,5-dihydroxy-1,3-benzene disulfonic acid, octyl isothiazolinone, picolinic acid, dipicolinic acid, 2-hydroxypyridine N-oxide, and mixtures thereof.

The weight ratio of chelant to fatty acid of formula I, fatty acid ester of formula I, or mixture thereof may be from about 1:30 to about 1:3, or from about 1:20 to about 1:5. Incorporation of one or more chelants into the compositions disclosed herein may provide additional potentiation benefits, further supplementing the activity of the fatty acid and/or fatty acid ester of formula I, particularly at greater composition pHs (e.g., pH about 3 to about 6 or pH about 4 to about 6).

Wipe or Pad

The present invention also relates to an article of manufacture comprising said composition, wherein the composition is comprised in a spray dispenser, or in a wipe or pad. The composition can be comprised on a wipe or pad. Such wipes and pads can be suitable for treating hard surfaces, such as found in the household, and the like. Suitable wipes can be fibrous. Suitable fibrous wipes can comprise polymeric fibres, cellulose fibres, and combinations thereof. Suitable cellulose-based wipes include kitchen wipes, and the like. Suitable polymeric fibres include polyethylene, polyester, and the like. Polymeric fibres can be spun-bonded to form the wipe. Methods for preparing thermally bonded fibrous materials are described in U.S. application Ser. No. 08/479,096 (Richards et al.), filed Jul. 3, 1995 (see especially pages 16-20) and U.S. Pat. No. 5,549,589 (Homey et al.), issued Aug. 27, 1996 (see especially Columns 9 to 10). Suitable pads include foams and the like, such as HIPE-derived hydrophilic, polymeric foam. Such foams and methods for their preparation are described in U.S. Pat. No. 5,550,167 (DesMarais), issued Aug. 27, 1996; and commonly assigned U.S. patent application Ser. No. 08/370,695 (Stone et al.), filed Jan. 10, 1995.

Methods of Use

The compositions disclosed herein may be used in a variety of applications and methods, including the treatment of hard surfaces, the treatment of soft inanimate surfaces, and the treatment of skin. The compositions may be used in the home to clean, sanitize, disinfect or sterilize hard surfaces, such as counters, sinks, restrooms, toilets, bath tubs, shower stalls, kitchen appliances, floors, windows, walls, furniture, phones, toys, drains, pipes, and the like. The compositions may also be used in commercial establishments, such as hotels, hospitals, care homes, eating establishments, fitness centers, schools, office buildings, department stores, and prisons, to clean, sanitize, disinfect, or sterilize equipment, tools, food and medical preparation areas (in addition to the surfaces mentioned above that are common to both homes and commercial establishments). The compositions disclosed herein may be used to treat indoor as well as outdoor surfaces and may also be used to sanitize, disinfect, or sterilize soft inanimate surfaces, such as carpets, area rugs, curtains, upholstery, and clothes, in both the home or in commercial settings.

The compositions may be used to treat bacteria, non-enveloped or enveloped viruses, fungi, spores, or allergens on surfaces or in the air. The compositions may also be used to purify contaminated water. The compositions may also be used in agricultural applications in the treatment of weeds, fruits, plants, and animals, including cattle and horses, as well as carcasses. The compositions may be used to disinfect or sanitize indoor or outdoor non-food, indirect food, or food contact agricultural premises, buildings, including animal housing, pens, feed troughs, greenhouses, storage containers and the like. The compositions may be used to sanitize or disinfect equipment used in non-food, indirect food, or food contact indoor and outdoor settings, including equipment used in green houses (with or without ornamental or food crops), feed handling, hatcheries, ice dispensing, processing livestock feeding, milk processing, milking, mushroom houses, poultry processing or handling, transport vehicles, and the like. The antimicrobial compositions, especially when formulated at a pH ranging from about 3.5 to about 6.0, may also be used to disinfect human skin.

Ready-to-use compositions may be housed in any container that allows for dispensing. Such containers may be metered to dispense a desired quantity or may include devices, such as caps, that allow the user to determine the level of dosing. Examples of containers include bottles, aerosols, pumps, and the like. Ready-to-use compositions may also be embedded in wipes or foams, such as melamine-formaldehyde foams. Such wipes may comprise woven and/or nonwoven substrates, where the substrates may include synthetic fibers, non-synthetic fibers, or mixtures of synthetic and non-synthetic fibers. As such, the wipe may optionally comprise cellulosic or non-cellulosic fibers, and, for high chemical resistance, may be in the form of a microfiber. The wipe may be a stand-alone or singly-formed substrate or a laminate of two or more substrates. Concentrates may be housed in any container as well. Concentrates may be dosed using mechanical or electrical pumps.

The present disclosure relates to an article of manufacture comprising the composition(s) disclosed herein in a spray dispenser or in a nonwoven substrate. The spray bottle may be a 2-chamber bottle in which, for example, the antimicrobial active is present in a first chamber and is separated from other formulation components present in the second chamber so as to mitigate or preclude reactivity of the antimicrobial active prior to use. Upon spraying, the contents of the 2 chambers are mixed together and sprayed as a uniform solution. The present disclosure also relates to a method of reducing the population of microorganism on a surface comprising the steps of applying an effective amount of the composition(s) disclosed herein to the surface and wiping the surface. The present disclosure also relates to a method of reducing population of microorganism on a surface comprising the steps of applying an effective amount of the composition(s) disclosed herein to the surface, where the composition contacts the surface for about 30 seconds to about 2 minutes, and wiping the surface.

### Examples

The bactericidal and fungicidal activity of the compositions of the present disclosure is quantified by the Association of Official Analytical Chemists (AOAC) Germicidal Spray Test (GST) Official Method 961.02, Germicidal Spray Products as Disinfectants (Official Methods of Analysis of the AOAC, 2009 Edition). Briefly, the GST is a carrier based method used to evaluate disinfection efficacy of aerosol/pump-based spray products and volatile liquid products for registration with regulatory agencies such as the US EPA and Health Canada. In this method, a series of glass slides (“carriers”) are inoculated with a representative test organism and dried for 30 minutes (>4 log inoculation). The carriers contacting the dried organism film are then sequentially treated with the spray product until thoroughly wet and are exposed for a finite contact time. After exposure, the carriers are sequentially transferred to a liquid subculture medium specifically selected to neutralize the test substance antimicrobial active and to recover any surviving test organism. The carriers are incubated and visually examined for the presence or absence of growth. Results are recorded as: number of carriers showing growth÷number of carriers tested. For example, a test result with 4 carriers showing growth out of 60 carriers tested would be recorded as 4/60; it is understood that a lower number of carriers showing growth is suggestive of a stronger performance. As such, comparisons may be made to differentiate the cidal efficacy of different compositions at a given contact time. In examples below, the exposure (contact) time for each experiment or group of experiments is provided.

For purposes of illustrating the benefits provided by the compositions disclosed herein, a series of 60 carrier tests vs. Gram (+) Staphylococcus aureus, a series of 60 carriers test vs. Gram (−) Pseudomonas aeruginosa, and a series of 30 carriers tests vs. fungi Trichophyton mentagrophytes are run. The benefits associated with the fatty acids and/or fatty acid esters of formula I are measured by comparing the performance of compositions with and without the fatty acid and/or fatty acid ester of formula I, and, in some cases, comparing the performance of compositions comprising fatty acid and/or fatty acid ester of formula I versus known compositions.

The compositions below are made up by mixing the components together. The concentration of each component in a given composition corresponds to the weight of the component, provided on an active basis, as a percent of the weight of the composition. The order in which components are added may be important. For example, the hydrogen peroxide may be added last and may be added at ambient conditions near room temperature (e.g., 20-23° C.) to avoid unintentional chemical reactivity. The components may be added in the following order: de-ionized water, then surfactant, then acid(s)—including the C₆₋₁₀ fatty acid (if present in the composition). The composition may be heated to 50°-60° C. to accelerate dissolution of components, especially succinic acid. Once cooled back to room temperature, the C₆₋₁₀ fatty acid ester (if present in the composition), the hydrogen peroxide, and a pH trimming solution (if one is used) are added under constant agitation to ensure solution homogeneity. All compositions in the examples below are clear or translucent solutions as made. The compositions are housed in amber HDPE bottles to protect from the effects of light during storage. All antimicrobial testing is conducted within about 2 months of product making or less, e.g., within about 1 month of product making. All samples are stored at ambient conditions (20-23° C.) prior to testing.

Just prior to testing, the compositions are diluted in either de-ionized water or in 400 ppm hardness AOAC synthetic water expressed as CaCO₃. The methodology is described in the Association of Official Analytical Chemists (AOAC), Official Method 960.09, Germicidal and Detergent Sanitizing Action of Disinfectants, Preparation of Synthetic Hard Water, in Official Methods of Analysis of the AOAC, 2005 Edition.

For the Germicidal Spray Test results, the following abbreviations are used:

SA=Staphylococcus aureus ATCC 6538
PA=Pseudomonas aeruginosa ATCC 15442
TM=Trichophyton mentagrophytes ATCC 9533

Dilutions are made on a volume basis. For example, a 1:30 dilution means that a ready-to-test solution is made by combining with 29 milliliters of water for each milliliter of the composition to be diluted, and a 1:50 dilution means that a ready-to-test solution is made by combining 49 milliliters of water for each milliliter of the composition to be diluted. DI H₂O denotes de-ionized water. Hard water here denotes 400 ppm AOAC synthetic water expressed as CaCO₃. Carrier results are reported as the number of carriers showing growth divided by the total number of carriers. This is abbreviated in the tables below as +ives/total.

### ABBREVIATIONS

C8 AS: Sodium octyl sulfate, tradename Stepanol® C-8 sulfate from the Stepan Company, supplied as a 33% active solution in water.
C10 AS: Sodium decyl sulfate, tradename Polystep® B-25 from the Stepan Company, supplied as a 38% solution in water.
C12 AS: Sodium lauryl sulfate, tradename Stepanol® WA-Extra, supplied as a 30% solution in water.
Succ.: succinic acid; a bio-based succinic acid from the Succinity Corporation supplied as a >99.5% powder.
Benzyl alcohol: Supplied by Lanxess Corporation as a ≧95% active raw material.
Hydrogen peroxide: Supplied by Sigma-Aldrich as a 30% solution in water or as a 35% active raw material, tradename Interox URM 35-D from Solvay Chemicals.
C8 glycerol ester: Capmul® 708G, a mixture of C8 mono glycerol ester (˜88%) and higher esters (˜12%); level of mono-dicaprylate ester≧99%.
C10 glycerol ester: Capmul® 9010G, a mixture of C10 mono glycerol ester (˜92%) and higher esters (˜8%); also contains ˜2.5% free glycerol.
Octanoic acid: C8-99K, ˜99.5% pure octanoic acid supplied by Procter & Gamble.
HPNO: 2-HydroxyPyridine N-oxide supplied by Sigma-Aldrich as a ≧96% active raw material
C8 hydroxamic: Octanohydroxamic acid supplied by Tokyo Chemical Industry Ltd. as a ≧99.0% active raw material.
Heptanoic Acid: Supplied by Tokyo Chemical Industry, Ltd. as ≧96% active raw material.
IDS: Tetrasodium Imino Disuccinate supplied by Lanxess as a 34% solution in water
NaOH: Sodium hydroxide, supplied as a 50% solution in water by Univar

### Compositions #1-4

Compositions #1-4 are tested to evaluate the potentiating effect of C₈ glycerol ester (also known as glycerol octanoate) on the biocidal activity of compositions comprising hydrogen peroxide. Germicidal spray test evaluations are performed in DI water and hard water conditions. Testing results are at a 1-minute exposure time.

Composition

| | #1* | #2 | #3 | #4 |
|---|---|---|---|---|
| Ingredient | (wt %) | (wt %) | (wt %) | (wt %) |
| C₈ AS | 6.0 | 6.0 | 6.0 | 6.0 |
| Succinic Acid | 5.0 | 5.0 | 5.0 | 5.0 |
| Hydrogen Peroxide | 4.5 | 4.5 | 4.5 | 4.5 |
| C₈ Glycerol Ester | — | 6.0 | 6.0 | 6.0 |
| HPNO | — | — | 0.5 | — |
| Acetic Acid | — | — | — | 4.5 |
| De-ionized Water | to 100 | to 100 | to 100 | to 100 |
| pH | 2.51 | 2.36 | 2.35 | 2.36 |

| | SA | SA | TM |
|---|---|---|---|
| | 1:30 wt % | 1:50 wt % | 1:20 wt % |
| | dilution | dilution | dilution |
| Composition # | DI H₂O | DI H₂O | DI H₂O |
| #1 | 60/60 | — | 30/30 |
| #2 | 0/60 | 0/60 | 0/30 |
| #3 | 0/60 | 1/60 | 0/30 |
| #4 | 0/60 | 0/60 | 0/30 |
| | SA | SA | TM |
| | 1:30 wt % | 1:50 wt % | 1:20 wt % |
| | dilution | dilution | dilution |
| Composition # | Hard H₂O | Hard H₂O | Hard H₂O |
| #1 | 60/60 | — | 30/30 |
| #2 | 0/60 | 0/60 | 0/30 |
| #3 | 0/60 | 0/60 | 1/30 |
| #4 | 0/60 | 0/60 | 0/30 |

*Comparative example

Composition #1, which lacks C₆₋₁₀ fatty acid and C₆₋₁₀ fatty acid ester, shows 60 out of 60 carrier failures. Composition #1 also shows 30 out of 30 carrier failures at a 1:20 dilution vs. Trichophyton mentagrophytes in both DI water and hard water conditions. Compositions #2, #3 and #4, which contain glycerol octanoate, show no more than 1 out of 60 carrier failures vs. Staphylococcus aureus at a 1:50 dilution and no more than 1 carrier failure vs. Trichophyton mentagrophytes at a 1:20 dilution in either hard DI water or hard water conditions. The results indicate that glycerol octanoate may be used to enhance the activity of hydrogen peroxide.

### Compositions #5-8

Compositions #5-8 are tested to evaluate the potentiating effect of C₁₀ glycerol ester or octanoic acid on the cidal activity of compositions comprising hydrogen peroxide. Germicidal spray test evaluations are performed in DI water and hard water conditions vs. Staphylococcus aureus and Trichophyton mentagrophytes. A known composition (#8) is also comparatively tested. Testing results are at a 1-minute exposure time.

Composition

| | #5 | #6 | #7 | #8* |
|---|---|---|---|---|
| Ingredient | (wt %) | (wt %) | (wt %) | (wt %) |
| C8 AS | 6.0 | 6.0 | 6.0 | 6.0 |
| Succinic Acid | 5.0 | 5.0 | 5.0 | 5.0 |
| Hydrogen Peroxide | 4.5 | 4.5 | 4.5 | 4.5 |
| C10 Glycerol Ester | 6.0 | 6.0 | 6.0 | — |
| Acetic Acid | — | 4.5 | — | — |
| Octanoic Acid | — | — | 4.0 | — |
| Benzyl Alcohol | — | — | — | 6.0 |
| De-ionized Water | to 100 | to 100 | to 100 | to 100 |
| pH | 2.40 | 2.39 | 2.32 | 2.35 |

| | SA | SA | TM |
|---|---|---|---|
| | 1:30 wt % | 1:50 wt % | 1:20 wt % |
| | dilution | dilution | dilution |
| Composition # | DI H₂O | DI H₂O | DI H₂O |
| #5 | 0/60 | — | 29/30 |
| #6 | 0/60 | 2/60 | 0/30 |
| #7 | — | 0/60 | 0/30 |
| #8 | 24/60 | 60/60 | 21/30 |
| | SA | SA | TM |
| | 1:30 wt % | 1:50 wt % | 1:20 wt % |
| | dilution | dilution | dilution |
| Composition # | Hard H₂O | Hard H₂O | Hard H₂O |
| #5 | 0/60 | 2/60 | 1/30 |
| #6 | 0/60 | 2/60 | 0/30 |
| #7 | — | 0/60 | 0/30 |
| #8 | 60/60 | 60/60 | 9/30 |

*Comparative example

Composition #5 is effective versus Staphylococcus aureus at a 1:50 dilution in hard water and versus Trichophyton mentagrophytes in hard water. The results versus Trichophyton mentagrophytes in DI water are improved by the addition of acetic acid to composition #5 (composition #6). Composition #5 and composition #6 are more cidally effective than composition #1, which lacks C₆₋₁₀ fatty acid and C₆₋₁₀ fatty acid ester, and composition #8, which includes benzyl alcohol but lacks C₆₋₁₀ fatty acid and C₆₋₁₀ fatty acid ester. Composition #7, which includes C₁₀ glycerol ester and octanoic acid, delivers very good cidal efficacy results.

### Compositions #9-12

Compositions #9-12 are made at pH 4.0±0.1, and tested to evaluate octanoic acid and glycerol octanoate in the presence and in the absence of a chelating agent. The chelating agent is either 2-hydroxypyridine-N-oxide (HPNO) or octanohydroxamic acid. Test results are at 1-minute exposure time.

Composition

| | #9 | #10 | #11 | #12 |
|---|---|---|---|---|
| Ingredient | (wt %) | (wt %) | (wt %) | (wt %) |
| C8 AS | 12.0 | 12.0 | 6.0 | 6.0 |
| Succinic Acid | 5.0 | 5.0 | 5.0 | 5.0 |
| Hydrogen Peroxide | 4.5 | 4.5 | 4.5 | 4.5 |
| Octanoic Acid | 4.0 | 4.0 | — | — |
| HPNO | — | 0.5 | — | — |
| C8 Glycerol Ester | — | — | 6.0 | 6.0 |
| C8 Hydroxamic | — | — | — | 0.5 |
| NaOH | — | — | 1.0 | 1.0 |
| De-ionized Water | to 100 | to 100 | to 100 | to 100 |
| pH | 4.02 | 4.02 | 3.93 | 4.02 |

| | SA | SA |
|---|---|---|
| | 1:50 wt % | 1:50 wt % |
| | dilution | dilution |
| Composition # | DI H₂O | Hard H₂O |
| #9 | 0/60 | 0/60 |
| #10 | 0/60 | 0/60 |
| #11 | 40/60 | 60/60 |
| #12 | 1/60 | 0/60 |

Compositions #9, #10, and #12 all pass the Germicidal Spray Test versus Staphylococcus aureus at a 1:50 dilution in DI water and hard water. Composition #11 is not effective versus Staphylococcus aureus when tested at these same conditions. The addition of a low concentration of octanohydroxamic acid to composition #11 (composition #12) substantially improves the effectiveness of the composition. These results indicate that effective antimicrobial compositions based on C₈ fatty acid and/or C₈ glycerol ester may be formulated at pH 4.0.

### Compositions #13-15

Compositions #13 is made at pH 2.5±0.1 while compositions 14-15 are made at pH 4.0±0.1. Citric acid and glutaric acid are used as the organic acid of choice, replacing succinic acid used in prior experiments. Test results are at 1-minute exposure time.

| | #13 | #14 | #15 |
|---|---|---|---|
| Ingredient | (wt %) | (wt %) | (wt %) |
| C8 AS | 12.0 | 12.0 | 9.0 |
| Citric Acid | 5.0 | 5.0 | — |
| Glutaric Acid | — | — | 6.0 |
| Hydrogen Peroxide | 4.5 | 4.5 | 4.5 |
| Octanoic Acid | 4.0 | 4.0 | 4.0 |
| IDS | — | — | 0.5 |
| NaOH | — | 2.0 | 0.8 |
| De-ionized Water | to 100 | to 100 | To 100 |
| pH | 2.52 | 4.02 | 4.00 |

| | SA | SA | TM | TM |
|---|---|---|---|---|
| | 1:50 wt % | 1:50 wt % | 1:20 wt % | 1:20 wt % |
| | dilution | dilution | dilution | dilution |
| Composition # | DI H₂O | Hard H₂O | DI H₂O | Hard H₂O |
| #13 | 0/60 | 0/60 | 0/30 | 0/30 |
| #14 | 0/60 | 0/60 | 0/30 | 0/30 |
| #15 | 0/60 | 0/60 | 0/30 | 0/30 |

Compositions #13-15 show complete cidal activity in the testing done. These results suggest the incentive compositions have flexibility with respect to selection of organic acid and with respect to pH choice,

### Compositions #16-17

Compositions #16-17 use heptanoic acid as the fatty acid source. Test results are at 1-minute exposure time.

| | #16 | #17 |
|---|---|---|
| Ingredient | (wt %) | (wt %) |
| C8 AS | 12.0 | 12.0 |
| Succinic Acid | 5.0 | — |
| Citric Acid | — | 6.0 |
| Glutaric Acid | — | — |
| Hydrogen Peroxide | 4.5 | 4.5 |
| Heptanoic Acid | 4.0 | 4.0 |
| NaOH | 0.1 | 0.5 |
| De-ionized Water | to 100 | to 100 |
| pH | 2.56 | 2.53 |

| | SA | SA | TM | TM |
|---|---|---|---|---|
| | 1:50 wt % | 1:50 wt % | 1:20 wt % | 1:20 wt % |
| | dilution | dilution | dilution | dilution |
| Composition # | DI H₂O | Hard H₂O | DI H₂O | Hard H₂O |
| #16 | 0/60 | 0/60 | 0/30 | 0/30 |
| #17 | 0/60 | 0/60 | 0/30 | 0/30 |

### Critical Micelle Concentration Measurements:

The Critical Micelle Concentrations of the compositions shown below are measured at pH 2.5±0.2 and/or pH 4.0±0.2. The measurements are made using a Beckman-Coulter workstation model Biomek FX. Measurements are conducted on the complete compositions below and the results are for the complete compositions tested. All of the compositions tested are compositions of the present disclosure. Additionally, all of the compositions have Critical Micelle Concentrations between about 100 ppm and about 2,500 ppm, or between about 300 ppm and about 1,500 ppm. The CMC values are not materially affected by the presence of hydrogen peroxide or by the presence of HPNO chelant.

| | | | CMC |
|---|---|---|---|
| Composition (wt %) | pH | Water | (ppm) |
| 6% C8 AS/5% Succ/6% C8 Glycerol Ester/6% | 2.5 | DI | 423 |
| H₂O₂ | | | |
| 6% C8 AS/5% Succ/6% C10 Glycerol Ester/6% | 2.5 | DI | 274 |
| H₂O₂ | | | |
| 6% C8 AS/5% Succ/6% C10 Glycerol Ester/6% | 2.5 | DI | 287 |
| H₂O₂/.5% HPNO | | | |
| 6% C10 AS/5% Succ/6% C10 Glycerol Ester/6% | 2.5 | DI | 262 |
| H₂O₂ | | | |
| 6% C10 AS/5% Succ/6% C10 Glycerol Ester/6% | 4.0 | DI | 589 |
| H₂O₂ | | | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition comprising from about 0.05% to about 40% of a surfactant comprising from about 6 to about 12 carbon atoms; from about 0.01% to about 40% of an acidifying agent; from about 0.01% to about 30% hydrogen peroxide; from about 0.01% to about 25% of a fatty acid of formula I, a fatty acid ester of formula I, or mixtures thereof:
R¹—C(O)O—R² (I)
wherein R¹ is selected from the group consisting of linear or branched, substituted or unsubstituted C₆-C₁₀ hydrocarbyl groups and R² is selected from the group consisting of H and linear or branched, substituted or unsubstituted C₁-C₆ hydrocarbyl groups; from about 15% to about 99.95% water, wherein said composition has a pH of from about 1.0 to about 6.0.

2. The composition according to claim 1, wherein said pH is from about 2.5 to about 5.0.

3. The composition according to claim 1, wherein said composition is an antimicrobial composition.

4. The composition according to claim 3, wherein said antimicrobial composition comprises an antimicrobial active which is hydrogen peroxide, at a level of from about 0.01% to about 8% of the composition.

5. The composition according to claim 1, wherein said composition has a critical micelle concentration from about 100 ppm to about 2,500 ppm.

6. The composition according to claim 1 wherein said surfactant is selected from the group consisting of C₈ glyceryl ether sulfonate, C₂-C₈ linear alkyl benzene sulfonate, C₆-C₁₂ alkyl sulfate, C₈-C₁₂ methyl ester sulfonate, C₈-C₁₂ fatty acid sulfonate, C₆-C₁₂ alkylethoxy carboxylate, C₆-C₁₂ alkylethoxy sulfate, C₈₋₁₀ dimethyl amine oxide, C₈ pyrrolidone, C₈ dimethyl betaine, C₈₋₁₀ alkyl polyglycoside, C₈₋₁₂ N,N-dimethyl-3-ammonio-1-propanesulfonate, and mixtures thereof.

7. The composition according to claim 1, wherein said composition comprises from about 0.03% to about 25%, preferably from about 0.05% to about 10%, of said acidifying agent.

8. The composition according to claim 1, wherein said acidifying agent is selected from the group consisting of formic acid, acetic acid, benzoic acid, malonic acid, citric acid, maleic acid, fumaric acid, hypochlorous acid, succinic acid, gluconic acid, glutaric acid, lactic acid, 2-ethyl-1-hexanoic acid, cinnamic acid, heptanoic acid, octanoic acid, nonanoic acid, peracetic acid, peroctanoic acid, undecylenic acid, and mixtures thereof.

9. The composition according to claim 1, wherein said composition is a concentrated composition comprising from about 1% to about 10% of said surfactant, wherein said surfactant comprises from about 6 to about 10 carbon atoms; from about 1% to about 10% of said acidifying agent; from about 1% to about 8% of said hydrogen peroxide; from about 1% to about 10% of said fatty acid of formula I, said fatty acid ester of formula I, or said mixtures thereof.

10. The composition according to claim 1, wherein said composition is a ready-to-use composition comprising from about 0.05% to about 0.50% of said surfactant, wherein said surfactant comprises from about 6 to about 10 carbon atoms; from about 0.05% to about 0.50% of said acidifying agent; from about 0.01% to about 0.50% of said hydrogen peroxide; from about 0.05% to about 0.50% of said fatty acid of formula I, said fatty acid ester of formula I, or said mixtures thereof.

11. The composition according to claim 1, wherein the composition comprises the fatty acid ester of formula I, wherein said fatty acid ester of formula I is selected from the group consisting of ethyl hexanoate, methyl octanoate, ethyl octanoate, glycerol octanoate, glycerol decanoate, and mixtures thereof.

12. The composition according to claim 1, wherein the composition comprises the fatty acid of formula I, wherein said fatty acid of formula I is selected from the group consisting of heptanoic acid, octanoic acid, and mixtures thereof.

13. The composition according to claim 1, wherein the weight ratio of said hydrogen peroxide to said fatty acid of formula I, said fatty acid ester of formula I, or said mixtures thereof is from about 0.2:1 to about 5:1.

14. The composition according to claim 1, wherein said composition comprises a C₆₋₁₀ fatty acid and a C₆₋₁₀ fatty peracid formed in-situ, wherein the ratio of C₆₋₁₀ fatty acid to C₆₋₁₀ fatty peracid is from about 10:1 to about 500:1.

15. The composition according to claim 1, wherein said composition comprises a solvent selected from the group consisting of ethanol, isopropanol, C₁-C₈ monoethylene glycol ether, C₁-C₈ diethylene glycol ether, C₁-C₈ triethylene glycol ether, C₁-C₆ monopropylene glycol ether, C₁-C₆ dipropylene glycol ether, C₁-C₆ tripropylene glycol ether, C₁-C₆ esters of formic acid, C₁-C₆ esters of acetic acid, C₁-C₆ esters of benzoic acid, C₁-C₆ esters of lactic acid, C₁-C₆ esters of 3-hydroxybutyric acid, C₁-C₆ amines, C₁-C₆ alkanol amines, and mixtures thereof.

16. The composition according to claim 1, wherein said acidifying agent is not a C₆₋₁₀ fatty acid.

17. An article of manufacture comprising the composition according to claim 1, wherein the composition is comprised in a spray dispenser, or in a wipe or pad.

18. A method of reducing the population of microorganism on a surface comprising the steps of applying an effective amount of the composition according to claim 1 to said surface and wiping said surface.

19. The method of reducing the population of microorganism on a surface according to claim 1, comprising the steps of applying an effective amount of the composition according to claim 1 to said surface, wherein said composition contacts said surface for about 30 seconds to about 2 minutes, and subsequently wiping said surface.

20. A method of treating a surface comprising the steps of: diluting a composition according to claim 1 with water in a weight ratio of the composition to water of from about 1:4 to about 1:250 such that an in-use composition is formed, and contacting said surface with said in-use composition.
